SEQUENCE LISTING (8 sequences)

SEQ ID NO: 1 - DNA, 24 bp, Artificial Sequence
  Synthetic construct
gaagacacccuaauguggcagctg

SEQ ID NO: 2 - DNA, 27 bp, Artificial Sequence
  Synthetic construct
cagcugccacauuagggugucuuccag

SEQ ID NO: 3 - DNA, 25 bp, Artificial Sequence
  Synthetic construct
acaacauugucugauaguagcuuga

SEQ ID NO: 4 - DNA, 27 bp, Artificial Sequence
  Synthetic construct
ucaagcuacuaucagacaauguuguuu

SEQ ID NO: 5 - DNA, 17 bp, Artificial Sequence
  Synthetic construct
cctcggaaccaggacct

SEQ ID NO: 6 - DNA, 20 bp, Artificial Sequence
  Synthetic construct
ttaatgcttccccacacctt

SEQ ID NO: 7 - DNA, 22 bp, Artificial Sequence
  Synthetic construct
atgtatgacaaaggacccttcc

SEQ ID NO: 8 - DNA, 21 bp, Artificial Sequence
  Synthetic construct
tcccttgcagagagtacatgg

(19) United States
(12) Patent Application Publication
(10) Pub. No.: US 2022/0088133 A1
(43) Pub. Date: Mar. 24, 2022

(54) Nanoliposome Compositions And Methods Of Treating Stroke

(71) Applicants: United States Government As Represented By The Department of Veterans Affairs, Washington, DC (US); Midwestern University, Downers Grove, IL (US)

(72) Inventors: Raymond Q. Migrino, Phoenix, AZ (US); Volkmar Weissig, Glendale, AZ (US)

(21) Appl. No.: 17/472,324

(22) Filed: Sep. 10, 2021

Related U.S. Application Data
(60) Provisional application No. 63/077,294, filed on Sep. 11, 2020.

(51) Int. Cl. A61K 38/17; A61K 31/7105; A61K 47/24; A61K 47/28; A61K 9/127; A61P 9/10; A61P 25/28

(52) U.S. Cl. CPC A61K 38/1761; A61K 38/1709; A61K 31/7105; A61P 25/28; A61K 47/28; A61K 9/1272; A61P 9/10; A61K 47/24

(57) ABSTRACT

Disclosed herein are compositions comprising nanoliposomes useful for the treatment and prevention of stroke.

### CROSS REFERENCE TO RELATED APPLICATIONS

This application claims the benefit of U.S. Provisional Application No. 63/077,294, filed Sep. 11, 2020. The content of this earlier filed application is hereby incorporated by reference herein in its entirety.

### STATEMENT REGARDING FEDERALLY FUNDED RESEARCH

This invention was made with government support under grant number BX003767 awarded by the U.S. Department of Veterans Affairs and grant number W81XWH-17-1-0473 awarded by the U.S. Department of Defense. The government has certain rights in the invention.

### INCORPORATION OF THE SEQUENCE LISTING

The present application contains a sequence listing that is submitted via EFS-Web concurrent with the filing of this application, containing the file name “37759_0346U2_SL.txt” which is 4,096 bytes in size, created on Sep. 10, 2021, and is herein incorporated by reference in its entirety.

### BACKGROUND

Stroke is the fourth leading cause of death in the U.S. (Appelros P, et al. Stroke. 2009; 40:1082-1090), and is the leading cause of serious long-term disability affecting more than 795,000 people in the United States every year (Benjamin et al., Circulation. 2017; 135:e229-e445). Medical treatment for ischemic stroke is limited to intravenous recombinant tissue plasminogen activator (IV r-tPA) which can reduce clot formation, but its use and effectiveness is limited because time dependency leads to low utilization rates in routine clinical practice. Invasive catheter-based extraction of clot (embolus or thrombus) requires specialized centers with advanced skills and specific timing, and even if successful, results often do not lead to full resolution or recovery of function (Albers et al., New England Journal of Medicine. 2018; 378:708-718). Thus, alternative methods and compositions to treat stroke are needed.

### SUMMARY

Disclosed herein are methods of treating stroke, the methods comprising administering to a subject a therapeutically effective amount of a composition comprising a nanoliposome, wherein the nanoliposome comprises a phospholipid, cholesterol, and a glycosphingolipid moiety.

Disclosed herein are methods of reducing or preventing medin-mediated injury in a subject, the methods comprising administering to the subject a therapeutically effective amount of a composition comprising a nanoliposome, wherein the nanoliposome comprises a phospholipid, cholesterol, and a glycosphingolipid moiety.

Disclosed herein are methods of reducing or preventing endothelial cell or vascular hypoxic/ischemic injury in a subject, the method comprising administering to the subject a therapeutically effective amount of a composition comprising a nanoliposome, wherein the nanoliposome comprises a phospholipid, cholesterol, and a glycosphingolipid moiety.

### BRIEF DESCRIPTION OF THE DRAWINGS

FIG. 1 shows cell viability under normal (physoxic) and hypoxic conditions. Human brain microvascular endothelial cells (HBMVECs) exposed to hypoxia (1% oxygen) for 20 hours showed reduced viability compared to cells exposed to physoxia. Treatment with monosialoganglioside-containing nanoliposomes (NLGM1) restored cell viability to cells exposed to hypoxia. N=4-5 each.

FIG. 2 shows the antioxidant stress response. Endothelial cells treated with monosialoganglioside-containing nanoliposomes (NLGM1) showed upregulation of antioxidant stress enzymes heme oxygenase-1 (HO-1), NADPH quinone dehydrogenase 1 (NQO1) and superoxide dismutase 1 (SOD1). Quantification of nuclear factor erythroid 2-related factor 2 (Nrf2) in nuclear components of endothelial cells show increased Nrf2 in NLGM1 treated cells versus controls. Nrf2 is a transcription factor that is a master regulator of production of antioxidant enzymes and protective mechanisms against hypoxic/ischemic stress and inflammatory stress. N=3-5 each.

FIG. 3 shows that human endothelial cells exposed to NLGM1 for 20 hours showed increased protein expression of p62 and LC3 II, proteins involved in autophagy and ubiquitination degradation processes.

FIGS. 4A-C show the changes in signal after intravenous (IV) injection of NLGM1. FIG. 4A shows background signal prior to IV injection of NLGM1 with incorporated fluorescent lipid, with bright fluorescent signal following IV injection into a mouse tail vein (FIG. 4B in the cerebrovasculature of a C57BL/6 mouse). FIG. 4C left panel shows the time course of the fluorescent signal in the brain with IV injection, with maximum signal 6 minutes post tail vein injection and signal persisting >2 hours. FIG. 4C right panel shows the time course of intraperitoneal (IP) injection showing maximum signal 198 minutes after injection and signal persisting >4 hours post injection.

FIGS. 5A-B show that C57BL/6 mice were exposed to middle cerebral artery occlusion (MCAo) to induce stroke and were treated with either saline control, or NLGM1 (10,000 μg/mL×4 doses). Neurologic impairment scoring in FIG. 5A showed significant reduction in neurologic impairment at 24 hours in mice treated with NLGM1 versus saline control (p=0.0154). Brain histopathology in FIG. 5B shows degree of brain infarct (TTC staining, pale regions) showing no damage in control mice without MCAo occlusion (normal), significant brain infarction in mice with MCAo treated with saline and reduced infarct size in mice with MCAo treated with NLGM1. Chart shows quantification of infarct volume showing significant reduction of infarct volume in mice treated with NLGM1 versus saline control (p=0.017, N=8, 7).

FIGS. 6A-D show hypoxic injury to human neuroblastoma and brain microvascular endothelial cells. FIG. 6A shows SH-SY5Y cells exposed to hypoxia for 20-hours reduce viability that was restored by treatment with NL (100 μg/ml or 300 μg/ml). FIGS. 6B-D show that hypoxia did not elicit any change in gene expression HO-1 (FIG. 6B), NQO1 (FIG. 6C) and SOD1 (FIG. 6D), but treatment with NL 300 μg/mL showed significant increase in gene expression of the antioxidant enzymes. FIG. 6E shows a similar response in endothelial cells with reduced viability with hypoxia and restoration of viability with NL treatment. *p<0.05, **p<0.01, ***p<0.001

FIGS. 7A-D show in vivo administration of NL. FIG. 7A shows the time course of IV-injected fluorophore-labelled NL. FIGS. 7B-D shows the time course of fluorescent signal in cerebral artery (red) and parenchymal brain regions (black) following IV and IP administration. Note that signal saturates at 256 arbitrary units (A.U.).

FIGS. 8A-D show that NL reduced stroke injury following middle cerebral artery occlusion (MCAO). FIG. 8A shows that mice treated with NL during MCAO showed less neurological impairment compared to saline-treated controls. FIG. 8B shows representative brain sections after TTC staining. FIGS. 8C-D show that mice who had MCAO treated with NL had smaller infarcts with a trend towards reduced brain edema in NL-treated mice.

### DETAILED DESCRIPTION

The present disclosure can be understood more readily by reference to the following detailed description of the invention, the figures and the examples included herein.

Before the present methods and gene expression panels are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment prior to the administering step.

As used herein, the term “comprising” can include the aspects “consisting of” and “consisting essentially of.”

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the term “gene” refers to a region of DNA encoding a functional RNA or protein. “Functional RNA” refers to an RNA molecule that is not translated into a protein. Generally, the gene symbol is indicated by using italicized styling while the protein symbol is indicated by using non-italicized styling.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

By “specifically binds” is meant that an antibody recognizes and physically interacts with its cognate antigen and does not significantly recognize and interact with other antigens; such an antibody may be a polyclonal antibody or a monoclonal antibody, which are generated by techniques that are well known in the art.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. In some aspects, preventing cerebrovascular disease, cell or tissue toxicity or hypoxic injury to endothelial cells, immune system activation, or neuroinflammation is intended.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a cerebrovascular disease” or “diagnosed with having had or at risk for having a stroke” means having been subjected to a physical examination by a person of skill, for example, a physician, and found to have a condition that can be diagnosed or can be treated by a composition that can prevent or inhibit cell or tissue toxicity or hypoxic injury to endothelial cells or reduce one or more proinflammatory or prothrombotic cytokines, or a combination thereof. As a further example, “diagnosed with a need for inhibiting medin protein expression” refers to having been subjected to a physical examination by a person of skill, for example, a physician, and found to have a condition characterized by increased levels of medin or other disease wherein inhibiting medin protein expression of a population of cells would be beneficial to the subject. Such a diagnosis can be in reference to a disorder, such as a cerebrovascular disease or stroke, as discussed herein.

As used herein, the phrase “identified to be in need of treatment for stroke,” or the like, refers to selection of a subject based upon need for treatment of the stroke. For example, a subject can be identified as having a need for treatment of stroke (based upon an earlier diagnosis by a person of skill and thereafter subjected to treatment for stroke. It is contemplated that the identification can, in some aspects, be performed by a person different from the person making the diagnosis. It is also contemplated, in a further aspect, that the administration can be performed by one who performed the diagnosis.

“Inhibit,” “inhibiting” and “inhibition” mean to diminish or decrease or reduce an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

As used herein, the term “biomarker” can refer to any molecular structure produced by a cell or organism having a molecular, biological or physical attribute that can be used to characterize a physiological or cellular state and that can be objectively measured to detect or define disease progression or predict or quantify therapeutic responses. A biomarker is a characteristic that is objectively measured and evaluated as an indicator of normal biologic processes, pathogenic processes, or pharmacologic responses to a therapeutic intervention. A biomarker may be expressed inside any cell or tissue; accessible on the surface of a tissue or cell; structurally inherent to a cell or tissue such as a structural component, secreted by a cell or tissue, produced by the breakdown of a cell or tissue through processes such as necrosis, apoptosis or the like; or any combination of these. A biomarker may be any protein, carbohydrate, fat, nucleic acid, catalytic site, or any combination of these such as an enzyme, glycoprotein, cell membrane, virus, cell, organ, organelle, or any uni- or multi-molecular structure or any other such structure now known or yet to be disclosed whether alone or in combination. In some aspects, the biomarker can be medin or a fragment thereof.

In some aspects, determining a level of expression of a biomarker can include quantitatively determining expression of a protein biomarker by routine methods known in the art. In some examples, an expression level of medin can be analyzed in a biological sample. Suitable biological samples include samples containing protein obtained from blood, urine or tissue from a subject, and/or protein obtained from one or more samples of control samples or subjects.

As used herein the term “effective amount” or “therapeutically effective amount” can refer to an amount of agent, such as a compositions comprising any of the nanoliposomes, compositions or pharmaceutical compositions described herein, that is sufficient to generate a desired response, such as reducing or eliminating a sign or symptom of a condition or disease, such as a cerebrovascular disease or stroke. In some aspects, the condition or disease can be characterized by overexpression of medin. In some aspects, the condition or disease is not characterized by overexpression of medin. Such signs or symptoms can include reduction in one or more proinflammatory or prothrombotic cytokines, preventing or reversing cell or tissue toxicity of a medin protein, preventing or reversing or reducing immune system activation, preventing, reversing or otherwise treating a cerebrovascular disease or stroke, increasing or upregulating one or more antioxidant stress enzymes or inhibiting the expression of medin protein within a cell.

Also, as used herein, the terms “effective amount”, “amount effective”, and “therapeutically effective amount” can refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. For example, in some aspects, an effective amount of a disclosed nanoliposome, composition or pharmaceutical composition is the amount effective to prevent or reduce cell or tissue toxicity of a medin protein, prevent or reverse or reduce immune system activation, reduce proinflammatory or prothrombotic cytokines, increase or upregulate one or more antioxidant stress enzymes and/or inhibit medin protein expression in a desired cell or population of cells. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a disclosed composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. In some aspects, a preparation can be administered in a “prophylactically effective amount”; that is, an amount effective for prevention of a disease or condition.

As used herein, “overexpression of medin” or “increased levels of medin” refers to the production of a gene product in subject or sample that exceeds levels of production in normal, control, or non-diseased subject (e.g. a subject with cerebrovascular disease or a subject with tissue toxicity or stroke caused by a medin protein). In some aspects, “overexpression of medin” or “increased levels of medin” refers to a level of expression of medin protein in a subject sufficient to cause toxicity. Similarly, an effective amount of compositions disclosed herein can inhibit or reduce or prevent or reverse cell or tissue toxicity caused by the increased expression of medin. Methods of measuring medin protein are known in the art and can include the Western blots described herein.

“Peptide” or “polypeptide” can refer to any chain of amino acids, regardless of length or posttranslational modification (such as glycosylation, methylation, ubiquitination, phosphorylation, or the like). In some aspects, a polypeptide is a medin polypeptide. Medin is a cleave product of parent protein MFGE8 or lactadherin (gene name is MFGE8). An amino acid sequence for medin is disclosed in Davies H A, et al. Scientific Reports 2017; 7, Article number 45224.

As used herein, “increase” or “upregulation” of one or more antioxidant stress enzymes refers to the production of a gene product or protein in a subject or a sample that exceeds levels of production in normal, control, or non-diseased subject. In some aspects, the one or more antioxidant stress enzymes can be heme-oxygenase 1 (HO1), NADPH quinone dehydrogenase (NQO1) and superoxide dismutase 1 (SOD1), or the amount in the nucleus of the gene regulator nuclear factor erythroid-2 related factor (Nrf2).

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described.

Publications cited herein and the materials for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert and applicants reserve the right to challenge the accuracy and pertinency of the cited documents. It will be clearly understood that, although a number of publications are referred to herein, such reference does not constitute an admission that any of these documents forms part of the common general knowledge in the art.

The present disclosure involves the use of nanoliposomes (which are small phospholipid particles and include but are not limited the medin-modifying nanoliposomes described herein) to prevent or reverse cell and tissue toxicity of the medin protein. The present disclosure involves the use of nanoliposomes to prevent or reverse cell and tissue toxicity that is not caused by the medin protein. Despite reports showing that medin is the most common and ubiquitous amyloid protein that accumulates in the vasculature with aging and reports by others that it induces toxicity to tissues (e.g., the vasculature), there is no known treatment to reverse its effects. Because medin is likely an important mediator of vascular aging, vascular inflammation and an important modulator of the interactions between age and cardiovascular risk factors leading to vascular dysfunction, the compositions and methods disclosed herein can be used for the treatment of atherosclerotic vascular disease, and ischemic neurologic diseases.

Medin is a 50 amino acid peptide that forms amyloid deposits; although it is not well-known, there is evidence that it may be the most common amyloid protein in humans (Haggqvist B, et al., Proceedings of the National Academy of Sciences of the United States of America 1999; 96:8669-8674; and Larsson A, et al. Biochem Biophys Res Commun 2007; 361:822-828). Disclosed herein are nanoliposomes which are small lipid (fat) particles that can be used in the methods disclosed herein. In some aspects, disclosed are nanoliposomes which are small lipid (fat) particles that can be used to change the biologic properties of medin and that can also be used to reverse or ameliorate the deleterious effects of medin. The nanoliposomes can be formulated either as lipid particles alone, or attached to other chemicals (serving as a carrier) to reverse medin effects. In some aspects, the nanoliposomes disclosed herein can be used to treat or prevent stroke or ameliorate one or more signs or symptoms of stroke. In some aspects, stroke or any of the signs of symptoms of stroke are not caused or otherwise attributed to medin.

Stroke remains a major cause of death and long-term disability (Chamorro A, et al. Lancet Neurol. 2016; 15:869-881). Prolonged ischemia and delayed reperfusion exacerbate injury (Nour M, et al. Interv Neurol. 2013; 1:185-99), highlighting the need to develop adjuvant therapies addressing reperfusion injury while extending the therapeutic window Saver J L, et al. Stroke. 2009; 40:2594-600). Nanoliposomes (<100 nm-sized phospholipids) comprising cholesterol, phosphatidylcholine and monosialoganglioside (NL) protected endothelial cells against oxidative stress induced by amyloidogenic light chain (Franco D A, et al. J Am Heart Assoc. 2016; 5) and medin proteins (Karamanova N, et al. J Am Heart Assoc. 2020; 9:e014810) through nuclear factor erythroid 2-related factor 2 (Nrf2)-mediated activation of antioxidant protective responses, with increased heme oxygenase-1 (HO-1), NAD(P)H quinone dehydrogenase 1 (NQO1) and superoxide dismutase-1 (SOD1). As described herein the efficacy of NL in mitigating hypoxic injury was evaluated by testing whether the NL disclosed herein can preserve viability of human neuroblastoma and brain microvascular endothelial cells exposed to hypoxia and that NL will reduce brain damage in mice subjected to middle cerebral artery occlusion (MCAO) stroke.

Compositions

Disclosed herein are compositions comprising nanoliposomes. In some aspects, the nanoliposomes can be medin-modifying nanoliposomes. As used herein, the term “medin-modifying nanoliposomes” can refer to a composition that can either prevent or reverse medin's adverse effects. For example, a medin-modifying nanoliposome can reverse endothelial cell immune activation caused by medin, inhibit NFκB activation, promote Nrf2-dependnet antioxidant responses, reduce or reverse increases in IL-8, IL-6, ICAM-1 or PAI-1, and restore endothelial cell viability. In some aspects, the nanoliposomes described herein can reduce, prevent endothelial cell or vascular hypoxic/ischemic injury in a cell. In some aspects, the nanoliposomes described herein can increase one or more of heme-oxygenase 1 (HO-1), NADPH quinone dehydrogenase (NQO1) or superoxide dismutase 1 (SOD1). In some aspects, the nanoliposomes described herein can increase gene expression of one or more of heme-oxygenase 1 (HO-1), NADPH quinone dehydrogenase (NQO1) or superoxide dismutase 1 (SOD1). In some aspects, the nanoliposomes described herein can increase gene expression of one or more of heme-oxygenase 1 (HO-1), NADPH quinone dehydrogenase (NQO1) or superoxide dismutase 1 (SOD1) compared to a reference sample or control. In some aspects, the nanoliposome described herein can comprise a phospholipid, cholesterol and a glycosphingolipid moiety.

The disclosed nanoliposomes at their core are nanoliposomes that have been modified. As such, the disclosure related to content, composition and method of making nanoliposomes can apply to any of the nanoliposomes disclosed herein. Nanoliposomes, including medin-modifying nanoliposomes, are composite structures made of phospholipids and may contain small amounts of other molecules. Though liposomes can vary in size from low micrometer range to tens of micrometers, nanoliposomes are typically in the lower size range. A nanoliposome has an aqueous solution core surrounded by a hydrophobic membrane, in the form of a lipid bilayer; hydrophilic solutes dissolved in the core cannot readily pass through the bilayer. Hydrophobic chemicals associate with the bilayer. A nanoliposome can be loaded with hydrophobic and/or hydrophilic molecules. To deliver the molecules to a site of action, the lipid bilayer can fuse with other bilayers such as the cell membrane, thus delivering the nanoliposome contents.

To deliver the nanoliposomes disclosed herein to a target in the brain, the nanoliposomes can be administered via multiple routes, including intravenous, intraperitoneal, subcutaneous, intramuscular or intranasally. By intranasal administration, the nanoliposome avoids the blood brain barrier and is absorbed through the olfactory and trigeminal nerves (e.g., direct nose-to-brain absorption). Alternatively, the nanoliposomes disclosed herein can reach the brain by attaching a biologically active ligand to the liposomal surface, wherein the biologically active ligand has receptors on the surface of the blood brain barrier. In some aspects, the biologically active ligand can be a peptide, an antibody or an antibody fragment, or small molecule. This approach utilizes existing active transport mechanisms (e.g., absorptive, carrier- or receptor-mediated transcytosis). Examples of biologically active ligands include but are not limited to glutathione, glucose, transferrin, lactoferrin, apolipoprotein E (ApoE), apolipoprotein J (ApoJ) and cell penetrating peptides (e.g., HIV-1 tat). In some aspects, the subject may have a disrupted blood brain barrier (e.g., caused by stroke) in which the nanoliposomes can exit the blood brain barrier into the brain parenchyma.

The choice of a nanoliposome preparation method depends on the following parameters: the physicochemical characteristics of the material to be entrapped (if any) and those of the nanoliposomal ingredients; the nature of the medium in which the lipid vesicles are dispersed; the effective concentration of the entrapped substance and its potential toxicity; additional processes involved during application/delivery of the vesicles; optimum size, polydispersity and shelf-life of the vesicles for the intended application; and, batch-to-batch reproducibility and possibility of large-scale production of safe and efficient liposomal products.

In some aspects, the nanoliposomes and medin-modifying nanoliposome described herein can refer to nanoscale lipid vesicles. Nanoliposomes have the same physical, structural, thermodynamic properties manufacturing and mechanism of formation as the liposomes. In some aspects, the nanoliposomes and medin-modifying nanoliposomes disclosed herein can have a diameter <100 nm, <75 nm, <50 nm, etc.

Disclosed herein are nanoliposomes comprising a phospholipid, cholesterol and a glycosphingolipid moiety. In some aspects, the phospholipid can be phosphatidylcholine. In some aspects, the phosphatidylcholine can be synthetic or natural (differentiated from each other by their fatty acid composition). In some aspects, the phospholipid can be a negatively charged phospholipid. In some aspects, the negatively charged phospholipid can be phosphatidic acid. In some aspects, the nanoliposomes and medin-modifying nanoliposomes can comprise phosphatidylcholine and cholesterol. Phospholipids can be used to make the nanoliposomes and medin-modifying nanoliposomes, and include but are not limited to synthetic lipids (e.g., 1,2-dipalmitoyl-sn-glycero-3-phosphocholine and ethyl-phosphatidylcholine) or natural lipids (e.g., phosphatidylcholine, sphingomyelin, and lecithin). Cholesterol can be added to the nanoliposomes and medin-modifying nanoliposomes during assembly to help maintain the stability of the membranes and reduce the permeability. The nanoliposomes and medin-modifying nanoliposomes can be functionalized. Modifications can include attachment of molecules to the exterior or encapsulation of molecules internally either in the aqueous core or lipid bilayers.

As used herein, the term “glycosphingolipid moiety” is a molecule comprising a glycosphingolipid (e.g., ceramide and oligosaccharide). Glycosphingolipids are a subtype of glycolipids containing the amino alcohol sphingosine. Glycosphingolipids can be considered as sphingolipids with an attached carbohydrate. In general, glycosphingolipids can be categorized into two groups: neutral glycosphingolipids (also called glycosphingolipids) and negatively charged glycosphingolipids. Examples of glycophingolipids include, but are not limited to cerebrosides, gangliosides and globosides. In some aspects, the glycosphingolipid moiety can be a cerebroside, a ganglioside, or a globoside. In some aspects, the glycosphingolipid moiety can be negatively charged.

In some aspects, the glycosphingolipid moiety can be a ganglioside. A ganglioside is a molecule composed of a glycosphingolipid with one or more sialic acids linked on the sugar chain. Gangliosides can be categorized by the number of sialic acids present, and include one NANA (“M”): GM1, GM2, and GM3; two NANAs (“D”): GD1a, GD1b, GD2, and GD3; three NANAs (“T”): GT1b and GT3; and four NANAs (“Q”): GQ1. In some aspects, the ganglioside can be GM1, GM2, GM3, GD1a, GD1b, GD2, GD3, GT1b, GT3 or GQ1. In some aspects, the ganglioside can be monosialoganglioside or GM1. In some aspects, the nanoliposomes can comprise phosphatidylcholine, cholesterol and GM1. Nanoliposomes composed of monosialoganglioside, phosphatidylcholine, and cholesterol can be referred to as GM1 ganglioside-containing nanoliposomes or monosialoganglioside-containing nanoliposomes or NLGM1. In some aspects, the molar ratio of phosphatidylcholine, cholesterol, and monosialoganglioside of the medin-modifying nanoliposome can be 70:25:5, respectively. In some aspects, the molar ratio of the phospholipid, cholesterol, and the glycosphingolipid of the nanoliposome can be 70:25:5, respectively. In some aspects, the phospholipid or the phosphatidylcholine can be 50, 60, 70, 80, or 90 or any number in between. In some aspects, the molar ratio of the cholesterol can be 5, 10, 15, 20, 25, or 30 or any number in between. In some aspects, the molar ratio of the glycosphingolipid can be about 1, 2, 3, 4, 5, 6, 7, 8, 9, 10, 11, 12, 13, 14, 15, 16, 17, 18, 19, or 20 or any number in between. In some aspects, the molar ratio of the phospholipid or phosphatidylcholine can be 70, 80, 90, or 95 or any number in between, the cholesterol can be 25, 20, 15, 10, 5, or 0 or any number in between, and the glycosphingolipid can be about 5, 6, 7, 8, 9, or 10. In some aspects, the phospholipid molar composition can range between 50-90%, the cholesterol molar composition can range between 5-30% and the glycosphingolipid molar composition can range between 1-20% in the combined composition.

In some aspects, the nanoliposomes and medin-modifying nanoliposomes can further comprise a cargo inside of the nanopliposome or medin-modifying nanoliposome or inside the lipid bilayer. In some aspects, the nanoliposomes and medin-modifying nanoliposomes can be loaded with cargo. In some aspects, the cargo can be a molecule. In some aspects, the cargo can be therapeutic cargo. Disclosed herein are compositions comprising a nanoliposomes or medin-modifying nanoliposomes and a therapeutic cargo. In some aspects, the nanoliposomes or medin-modifying nanoliposomes can comprise a phospholipid and cholesterol. In some aspects, the phospholipid can be phosphatidylcholine. In some aspects, the therapeutic cargo can be a glycophingolipid moiety. In some aspects, the glycophingolipid moiety can be a cerrebroside, a ganglioside or a globoside. In some aspects, the therapeutic cargo can be a ganglioside. In some aspects, the ganglioside can be GM1, GM2, GM3, GD1a, GD1b, GD2, GD3, GT1b, GT3 or GQ1. In some aspects, the ganglioside can be monosialoganglioside or GM1. In some aspects, the therapeutic cargo can be clusterin. In some aspects, the clusterin can be secretory clusterin. In some aspects, the therapeutic cargo can be secretory clusterin (sCLU). In some aspects, the therapeutic cargo can be apolipoprotein J. Examples of therapeutic cargoes include but not limited to apolipoproteins such as apolipoproteins A1 and E, peptides, antibodies, antibody fragments, nucleic acids such as siRNA, aptamers, small interfering RNAs (siRNAs), short hairpin RNAs (shRNAs), and mitochondrially targeted antioxidants such as vitamin E. In some aspects, any of the compositions disclosed herein can further comprise a pharmaceutically acceptable carrier.

In some aspects, the nanoliposomes or medin-modifying nanoliposomes can comprise phosphatidylcholine, cholesterol, a ganglioside, and a therapeutic cargo. In some aspects, the therapeutic cargo can be a ganglioside. In some aspects, the ganglioside can be GM1, GM2, GM3, GD1a, GD1b, GD2, GD3, GT1b, GT3 or GQ1. In some aspects, the ganglioside can be monosialoganglioside or GM1. In some aspects, the therapeutic cargo can be clusterin. In some aspects, the clusterin can be secretory clusterin. In some aspects, the therapeutic cargo can be secretory clusterin (sCLU). In some aspects, the therapeutic cargo can be apolipoprotein J. Examples of therapeutic cargoes include but not limited to apolipoproteins such as apolipoproteins A1 and E, peptides, antibodies, antibody fragments, nucleic acids such as siRNA, aptamers, small interfering RNAs (siRNAs), short hairpin RNAs (shRNAs), and mitochondrially targeted antioxidants such as vitamin E. In some aspects, any of the compositions disclosed herein can further comprise a pharmaceutically acceptable carrier.

Disclosed herein are compositions comprising any of the nanoliposomes or medin-modifying nanoliposomes described herein. In some aspects, the compositions can further comprise a therapeutic cargo. In some aspects, the therapeutic cargo can be clusterin. In some aspects, the clusterin can be secretory clusterin. In some aspects, the therapeutic cargo can be secretory clusterin (sCLU). In some aspects, the therapeutic cargo can be apolipoprotein J. Examples of therapeutic cargoes include but not limited to apolipoproteins such as apolipoproteins A1 and E, peptides, antibodies, antibody fragments, nucleic acids such as siRNA, aptamers, small interfering RNAs (siRNAs), short hairpin RNAs (shRNAs), and mitochondrially targeted antioxidants such as vitamin E. In some aspects, any of the compositions disclosed herein can further comprise a pharmaceutically acceptable carrier.

Disclosed herein are compositions comprising any of the compositions described herein. In some aspects, any of the nanoliposomes or medin-modifying nanoliposomes described herein or any of the compositions described herein can be co-formulated with one or more therapeutic agents. In some aspects, the one or more therapeutic agents can be therapeutic agents that are used to treat to an ischemic cerebrovascular disease, stroke, or endothelial cell or vascular hypoxic/ischemic injury. In some aspects, the one or more therapeutic agents that can be used to treat an ischemic cerebrovascular disease, stroke, or endothelial cell or vascular hypoxic/ischemic injury can be an antioxidant, an anti-inflammatory agent, an anti-apoptotic agent and an autophagy-modifying agent. In some aspects, the compositions can be formulated for oral, subcutaneous, intrathecal, intramuscular, inhalation, or intravenous administration.

In some aspects, any of the compositions disclosed herein can further comprise a medin binding molecule. In some aspects, the medin binding molecule comprises a medin binding moiety. In some aspects, the medin binding moiety is capable of specifically binding to medin or a fragment thereof. In some aspects, the binding moiety can be any material that can selectively form a stable complex or a covalent bond with medin or a fragment thereof. In some aspects, the binding moiety can be a peptide, antibody, small molecule, or a nucleic acid. In some aspects, the antibody can be a single chain antibody (scFv) or a Fab fragment, human, chimeric or humanized or a biologically active variant thereof, a monoclonal antibody, or a polyclonal antibody.

As used herein, the term “medin binding moiety” can be used to described a portion or a component of the medin binding molecule which binds to medin selectively and is used in herein to target the protein medin in a cell, tissue or sample from a subject (e.g. blood), for example, which is overexpressed or hyperexpressed in a cerebrovascular disease compared to normal tissue.

As noted above, any of the compositions as disclosed herein, can include an antibody or a biologically active variant thereof (e.g., an antibody the specifically binds to medin). As is well known in the art, monoclonal antibodies can be made by recombinant DNA. DNA encoding monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques.

In vitro methods are also suitable for preparing monovalent antibodies. As it is well known in the art, some types of antibody fragments can be produced through enzymatic treatment of a full-length antibody. Digestion of antibodies to produce fragments thereof, particularly, Fab fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen. Antibodies incorporated into the present compositions can be generated by digestion with these enzymes or produced by other methods.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment can be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment.

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen binding portion of an antibody) which contains a portion of an antigen binding site from a non-human (donor) antibody integrated into the framework of a human (recipient) antibody.

The Fv region is a minimal fragment containing a complete antigen-recognition and binding site consisting of one heavy chain and one light chain variable domain. The three CDRs of each variable domain interact to define an antigen-biding site on the surface of the Vh-Vl dimer. Collectively, the six CDRs confer antigen-binding specificity to the antibody. As well known in the art, a “single-chain” antibody or “scFv” fragment is a single chain Fv variant formed when the VH and Vl domains of an antibody are included in a single polypeptide chain that recognizes and binds an antigen. Typically, single-chain antibodies include a polypeptide linker between the Vh and Vl domains that enables the scFv to form a desired three-dimensional structure for antigen binding.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies can also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody.

Methods for humanizing non-human antibodies are well known in the art. For example, humanized antibodies can be generated by substituting rodent CDRs or CDR sequences for the corresponding sequences of a human antibody. Methods that can be used to produce humanized antibodies are also well known in the art.

Pharmaceutical Compositions

As disclosed herein, are pharmaceutical compositions comprising any of the nanoliposomes or medin-modifying nanoliposomes described herein or any of the compositions described herein. In some aspects, the pharmaceutical compositions disclosed herein can further comprise an aqueous solution. In some aspects, the aqueous solution can comprise the nanoliposomes or the medin-modifying nanoliposomes. In some aspects, the aqueous solution of the pharmaceutical composition can be adjusted to a human physiological pH. In some aspects, the pharmaceutical composition can be formulated for intravenous administration. In some aspects, the pharmaceutical composition can be formulated for oral administration. In some aspects, the pharmaceutical composition can be formulated for subcutaneous, intramuscular, or intranasal administration. In some aspects, the pharmaceutical composition can be formulated for intrathecal administration. The compositions of the present disclosure also contain a therapeutically effective amount of any of the nanoliposomes or medin-modifying nanoliposomes as described herein. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The pharmaceutical compositions disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the nanoliposomes or medin-modifying nanoliposomes. Thus, compositions can be prepared for parenteral administration that includes nanoliposomes or medin-modifying nanoliposomes suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. In some aspects, a dry pharmaceutical composition can be formed by drying the pharmaceutical composition. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some aspects, the disclosed pharmaceutical composition or any of the compositions disclosed herein or any of the nanoliposomes or medin-modifying nanoliposomes disclosed herein can be administered in combination with one or more therapeutic agents. In some aspects, the one or more therapeutic agents can be used to treat ischemic cerebrovascular disease, stroke or reduce or prevent endothelial cell or vascular hypoxic/ischemic injury. Dosing with any of the one or more therapeutic agents (and other pharmaceutical compositions) is known in the art and can be altered to be used in combination with the disclosed pharmaceutical compositions or any of the compositions disclosed herein or any of the nanoliposomes or medin-modifying nanoliposomes disclosed herein by one of skill in the art in light of this disclosure. The one or more therapeutic agents can be given concurrently with, prior to or after the administration of any of the disclosed pharmaceutical compositions, or any of the compositions disclosed herein or any of the nanoliposomes or medin-modifying nanoliposomes disclosed herein.

Methods of Treatment

Disclosed herein are methods of treating stroke. In some aspects, the methods can comprise administering to a subject with stroke, at risk or has been identified as being at risk of stroke and/or at risk of elevated stroke mortality a therapeutically effective amount of any of the medin-modifying nanoliposomes, nanoliposomes, pharmaceutical compositions, or compositions disclosed herein. Disclosed herein are methods of ameliorating one or more symptoms of stroke. In some aspects, the methods can comprise administering to a subject with stroke, at risk or has been identified as being at risk of stroke and/or at risk of elevated stroke mortality a therapeutically effective amount of any of the medin-modifying nanoliposomes, nanoliposomes, pharmaceutical compositions or compositions disclosed herein.

Disclosed herein methods of reducing or preventing endothelial cell or vascular hypoxic/ischemic injury. In some aspects, the methods can comprise administering to a subject with an endothelial cell or vascular hypoxic/ischemic injury or at risk for an endothelial cell or vascular hypoxic/ischemic injury a therapeutically effective amount of any of the medin-modifying nanoliposomes, nanoliposomes, pharmaceutical compositions, or compositions disclosed herein. In some aspects, the methods can comprise administering to a subject with an endothelial cell or vascular hypoxic/ischemic injury or at risk for an endothelial cell or vascular hypoxic/ischemic injury a therapeutically effective amount of any of the medin-modifying nanoliposomes, nanoliposomes, pharmaceutical compositions, or compositions disclosed herein.

Disclosed herein are methods of preventing or reversing cell or tissue toxicity of a medin protein. In some aspects, the methods can comprise administering to in a subject with an ischemic cerebrovascular disease, endothelial cell or vascular hypoxic/ischemic injury or stroke a therapeutically effective amount of any of the nanoliposomes, medin-modifying nanoliposomes, any of the compositions or any of the pharmaceutical compositions disclosed herein.

Disclosed herein are methods of reducing or preventing medin-mediated injury in a subject. In some aspects, the methods can comprise administering to in a subject with an ischemic cerebrovascular disease, endothelial cell or vascular hypoxic/ischemic injury or stroke a therapeutically effective amount of any of the nanoliposomes, medin-modifying nanoliposomes, or any of the compositions or pharmaceutical compositions disclosed herein.

Disclosed herein are method of increasing one or more antioxidant enzymes. In some aspects, the methods can comprise administering to in a subject with an ischemic cerebrovascular disease, endothelial cell or vascular hypoxic/ischemic injury or stroke a therapeutically effective amount of any of the nanoliposomes, medin-modifying nanoliposomes, or any of the compositions or pharmaceutical compositions disclosed herein. In some aspects, the one or more antioxidant enzymes can be heme-oxygenase 1 (HO-1), NADPH quinone dehydrogenase (NQO1), superoxide dismutase 1 (SOD1) or a combination thereof. In some aspects, the one or more antioxidant enzymes can be catalase, clutathione peroxidase, peroxiredoxin I and II, thioredoxin, myeloperoxidase, thioredoxin reductase, or a combination thereof. In some aspects, the one or more antioxidant enzymes can be heme-oxygenase 1 (HO-1), NADPH quinone dehydrogenase (NQO1), superoxide dismutase 1 (SOD1), catalase, clutathione peroxidase, peroxiredoxin I and II, thioredoxin, myeloperoxidase, thioredoxin reductase, or a combination thereof. In some aspects, the nanoliposomes described herein can increase gene expression of one or more of heme-oxygenase 1 (HO-1), NADPH quinone dehydrogenase (NQO1) or superoxide dismutase 1 (SOD1). In some aspects, the nanoliposomes described herein can increase gene expression of one or more of heme-oxygenase 1 (HO-1), NADPH quinone dehydrogenase (NQO1) or superoxide dismutase 1 (SOD1) compared to a reference sample or control.

Disclosed herein are method of increasing one or more transcription factors. In some aspects, the methods can comprise administering to in a subject with an ischemic cerebrovascular disease, endothelial cell or vascular hypoxic/ischemic injury or stroke a therapeutically effective amount of any of the nanoliposomes, medin-modifying nanoliposomes, or any of the compositions or pharmaceutical compositions disclosed herein. In some aspects, the one or more transcription factors can regulate one or more antioxidant proteins. In some aspects, increasing one or more transcription factors can reduce or prevent oxidative damage or protect against oxidative damage. In some aspects, the one or more transcription factors can be nuclear factor erythroid 2-related factor 2 (Nrf2).

Disclosed herein are methods of preventing or reversing or reducing immune system activation. In some aspects, the methods can comprise administering to a subject with a ischemic cerebrovascular disease, stroke or endothelial cell or vascular hypoxic/ischemic injury a therapeutically effective amount of any of the nanoliposomes, any of the medin-modifying nanoliposomes, any of the compositions or any of the pharmaceutical compositions disclosed herein. In some aspects, the immune system activation can be associated with an increase in one or more proinflammatory or prothrombotic cytokines. In some aspects, the one or more proinflammatory cytokines can be IL-8 or IL-6. In some aspects, the one or more prothrombotic cytokines can be ICAM-1 or PAI-1.

Disclosed herein methods of reducing one or more proinflammatory or prothrombotic cytokines or pro-inflammatory transcription factors. In some aspects, the methods can comprise administering to a subject with a cerebrovascular disease or an endothelial cell or vascular hypoxic/ischemic injury a therapeutically effective amount of any of the nanoliposomes, any of the medin-modifying nanoliposomes, any of the compositions or any of the pharmaceutical compositions disclosed herein. In some aspects, the one or more proinflammatory cytokines can be IL-1β, IL-8, TNF-α or IL-6. In some aspects, the one or more prothrombotic cytokines can be ICAM-1, VCAM-1, MCP, caspase-1 or PAI-1. In some aspects, the pro-inflammatory transcription factors can be nuclear factor kappa light chain enhancer of activated B cells (NFκB). In some aspects, the methods described herein can also reduce one or more inflammasomes. In some aspects, the one or more inflammasomes can be caspase-1

Disclosed herein are methods of preventing or reversing or reducing neuroinflammation, the method comprising administering to in a subject with an ischemic cerebrovascular disease, stroke or an endothelial cell or vascular hypoxic/ischemic injury a therapeutically effective amount of any of the nanoliposomes, any of the medin-modifying nanoliposomes, any of the compositions or any of the pharmaceutical compositions disclosed herein.

Disclosed herein are methods of increasing prosurvival or autophagic pathways, the method comprising administering to in a subject with an ischemic cerebrovascular disease, stroke or an endothelial cell or vascular hypoxic/ischemic injury a therapeutically effective amount of any of the nanoliposomes, any of the medin-modifying nanoliposomes, or any of the compositions or pharmaceutical compositions disclosed herein. In some aspects, one or more prosurvival or autophagic pathways components are increased. In some aspects, one or more prosurvival or autophagic pathways components can be sequestasome (p62), LC3-II, autophagosome, autolysosome or a combination thereof.

Disclosed herein are methods of increasing nitric oxide bioavailability, the method comprising administering to in a subject with an ischemic cerebrovascular disease, stroke or an endothelial cell or vascular hypoxic/ischemic injury a therapeutically effective amount of any of the nanoliposomes, any of the medin-modifying nanoliposomes, or any of the compositions or pharmaceutical compositions disclosed herein. In some aspects, one or more of the nitric oxide pathways can be activated or increased. In some aspects, one or more nitric oxide pathway components can be activated or increased. In some aspects, the one or more nitric oxide pathway components can be nitric oxide, endothelial nitric oxide synthase (eNOS), eNOS coupling, peroxynitrite or a combination thereof.

Disclosed herein are methods of decreasing prothrombotic or procoagulant processes or factors, the methods comprising administering to in a subject with an ischemic cerebrovascular disease, stroke or an endothelial cell or vascular hypoxic/ischemic injury a therapeutically effective amount of any of the nanoliposomes, any of the medin-modifying nanoliposomes, or any of the compositions or pharmaceutical compositions disclosed herein. In some aspects, the one or more prothrombotic or procoagulant factors can be plasminogen activator inhibitor-1 (PAI-1), tissue factor, thrombomodulin, von Willebrand factor, intrinsic and extrinsic coagulation pathways, or a combination.

Disclosed herein are methods of protecting endothelial cells exposed to amyloid insults comprising contacting the endothelial cells with an effective amount of a composition to protect the endothelial cells exposed to amyloid insults, wherein the composition comprises a nanoliposome, wherein the nanoliposome comprises a phospholipid, cholesterol, and a glycosphingolipid moiety. In some aspects, the endothelial cells are in a subject.

Disclosed herein are methods of protecting endothelial cells exposed to amyloid insults in a subject comprising administering to the subject a therapeutically effective amount of a composition comprising a nanoliposome, wherein the nanoliposome comprises a phospholipid, cholesterol, and a glycosphingolipid moiety, thereby protecting the endothelial cells exposed to amyloid insults in the subject.

A method of preserving cell viability following a hypoxic injury comprising contacting the cells with an effective amount of a composition to preserve cell viability following a hypoxic injury, wherein the composition comprises a nanoliposome, wherein the nanoliposome comprises a phospholipid, cholesterol, and a glycosphingolipid moiety, wherein the viability of the cells is higher than similar cells following a hypoxic injury not contacted with the composition. In some aspects, the cells are in a subject.

In some aspects, the stroke can be an ischemic stroke, a hemorrhagic stroke, a transient ischemic attack, cryptogenic stroke or vascular dementia. In some aspects, the subject has an ischemic injury caused by atherosclerotic cerebrovascular disease. In some aspects, the subject does not have increased or elevated levels of medin.

In some aspects, the subject can be identified in need of treatment before the administration step. In some aspects, the subject can be identified as having an increased or elevated level of medin protein compared to a control subject or control sample. In some aspects, the subject can be identified as not having an increased or elevated level of medin protein compared to a control subject or control sample. In some aspects, the subject can be a human.

The pharmaceutical compositions described above can be formulated to include a therapeutically effective amount of any of the nanoliposomes disclosed herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to an ischemic cerebrovascular disease, stroke or endothelial cell or vascular hypoxic/ischemic injury.

The pharmaceutical compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with an ischemic cerebrovascular disease, stroke or endothelial cell or vascular hypoxic/ischemic injury in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effective amount includes amounts that provide a treatment in which the onset or progression of the ischemic cerebrovascular disease, stroke or the endothelial cell or vascular hypoxic/ischemic injury is delayed, hindered, or prevented, or the ischemic cerebrovascular disease, stroke or the endothelial cell or vascular hypoxic/ischemic injury or a symptom of the ischemic cerebrovascular disease, stroke or the endothelial cell or vascular hypoxic/ischemic injury is ameliorated. One or more of the symptoms can be less severe. Recovery can be accelerated in an individual who has been treated.

In some aspects, the nanoliposomes disclosed herein are capable of persisting in the circulation (e.g., cerebrovascular circulation) for longer periods of time than other nanoliposomes.

Disclosed herein, are methods of treating a patient with an ischemic cerebrovascular disease, stroke or the endothelial cell or vascular hypoxic/ischemic injury. In some aspects, the patient can have one or more of ischemic cerebrovascular disease, stroke or the endothelial cell or vascular hypoxic/ischemic injury or a combination thereof. In some aspects, the patient can be at risk of one or more of ischemic cerebrovascular disease, stroke or the endothelial cell or vascular hypoxic/ischemic injury or a combination thereof. In some aspects, the patient can have one or more of ischemic cerebrovascular disease, stroke or the endothelial cell or vascular hypoxic/ischemic injury or a combination thereof and be at risk of one or more ischemic cerebrovascular disease, stroke or the endothelial cell or vascular hypoxic/ischemic injury or a combination thereof.

Amounts effective for this use can depend on the severity of the ischemic cerebrovascular disease, stroke or the endothelial cell or vascular hypoxic/ischemic injury and the weight and general state and health of the subject, but generally range from about 0.05 μg to about 1000 μg (e.g., 0.5-1000 μg) of an equivalent amount of the nanoliposome or medin-modifying nanoliposome per dose per subject. Suitable regimes for initial administration and booster administrations are typified by an initial administration followed by repeated doses at one or more hourly, daily, weekly, or monthly intervals by a subsequent administration. For example, a subject can receive any of the pharmaceutical compositions, compositions, nanoliposomes or medin-modifying nanoliposomes one or more times per week (e.g., 2, 3, 4, 5, 6, or 7 or more times per week). For example, a subject may receive 0.1 to 2,500 μg (e.g., 2,000, 1,500, 1,000, 500, 100, 10, 1, 0.5, or 0.1 μg) dose per week. A subject can also receive a nanoliposomes or medin-modifying nanoliposomes in the range of 0.1 to 3,000 μg per dose once every two or three weeks. A subject can also receive 2 mg/kg every week (with the weight calculated based on the weight of the nanoliposomes or medin-modifying nanoliposomes).

The total effective amount of a nanoliposomes or medin-modifying nanoliposomes in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

The therapeutically effective amount of one or more of the therapeutic agents present within the compositions described herein and used in the methods as disclosed herein applied to mammals (e.g., humans) can be determined by one of ordinary skill in the art with consideration of individual differences in age, weight, and other general conditions (as mentioned above).

Also disclosed herein are methods of detecting a medin protein or a fragment thereof in a sample. In some aspects, the methods can comprise contacting a medin-modifying nanoliposome as described herein or a nanoliposome as described herein comprising a medin binding moiety with the sample. In some aspects, the medin-modifying nanoliposome or the nanoliposome comprising a medin binding moiety can comprise a detectable label. In some aspects, the sample can comprise a detectable level of the medin protein. In some aspects, the methods can comprise detecting binding of the medin-modifying nanoliposome or the nanoliposome comprising a medin binding moiety to the medin protein.

In some aspects, the detectable label can be any detectable moiety. For example, the detectable label can be fluorescein, HA tag, Gst-tag, EGFP-tag, FLAG™ tag or biotin.

Epitope tags are short stretches of amino acids to which a specific antibody can be raised, which in some aspects allows one to specifically identify and track the tagged protein that has been added to a living organism or to cultured cells. Detection of the tagged molecule can be achieved using a number of different techniques. Examples of such techniques include: immunohistochemistry, immunoprecipitation, flow cytometry, immunofluorescence microscopy, ELISA, immunoblotting (“Western blotting”), and affinity chromatography. Epitope tags add a known epitope (e.g., antibody binding site) on the subject protein, to provide binding of a known and often high-affinity antibody, and thereby allowing one to specifically identify and track the tagged protein that has been added to a living organism or to cultured cells. Examples of epitope tags include, but are not limited to, myc, T7, GST, GFP, HA (hemagglutinin), V5 and FLAG tags. The first four examples are epitopes derived from existing molecules. In contrast, FLAG is a synthetic epitope tag designed for high antigenicity (see, e.g., U.S. Pat. Nos. 4,703,004 and 4,851,341). Epitope tags can have one or more additional functions, beyond recognition by an antibody. The sequences of these tags are described in the literature and well known to the person of skill in art.

In some aspects, the disclosed methods and compositions comprise an epitope-tag wherein the epitope-tag has a length of between 6 to 15 amino acids. In an alternative aspect, the epitope-tag has a length of 9 to 11 amino acids.

As described herein, the term “immunologically binding” is a non-covalent form of attachment between an epitope of an antigen (e.g., the epitope-tag) and the antigen-specific part of an antibody or fragment thereof. Antibodies are preferably monoclonal and must be specific for the respective epitope tag(s) as used. Antibodies include murine, human and humanized antibodies. Antibody fragments are known to the person of skill and include, amongst others, single chain Fv antibody fragments (scFv fragments) and Fab-fragments. The antibodies can be produced by regular hybridoma and/or other recombinant techniques. Many antibodies are commercially available.

In some aspects, the methods can further comprise measuring (e.g., quantifying) the amount of the detected medin protein in the sample. In some aspects, the methods can further comprise comparing the amount of the detected medin protein in the sample with a control sample. In some aspects, the sample can be blood, urine or tissue. In some aspects, the amount of the detected medin protein in the sample can be higher than the amount of the medin protein in the control sample indicating an increased risk for developing or having a cerebrovascular disease or an aging-related degenerative disease. In some aspects, the amount of the detected medin protein in the sample can be lower than or about equal to the amount of the medin protein in the control sample indicating a reduced risk for developing or having an ischemic cerebrovascular disease, stroke or the endothelial cell or vascular hypoxic/ischemic injury.

### EXAMPLES

### Example 1: Modulation of Endothelial Cell Hypoxic Injury by Amyloidogenic Medin: Implications for Aging-Related Cerebrovascular Disease

Introduction. Medin is a 50-amino acid amyloidogenic peptide that accumulates in the vasculature with aging (Larrson A, et al. Amyloid 2006; 13:78-85). It was recently shown that medin is present in cerebral arteries of elderly brain donors with greater amount in vascular dementia versus cognitively normal subjects (Karamanova N, et al. J Am Heart Assoc 2020; 9:e014810). Medin was also shown to cause endothelial dysfunction and inflammatory activation (Karamanova N, et al. J Am Heart Assoc 2020; 9:e014810; and Migrino R Q, et al. Cardiovasc Res 2017; 113: 1389-1402), the former related to induction of oxidative stress. These findings suggest that medin may be an important mediator in cerebrovascular disease and vascular dementia. The effect of medin on endothelial cell (EC) function in setting of hypoxic injury is not known. Nanoliposomes are <100 nM phospholipid-containing particles. It was also recently shown that the nanoliposome, GM1L, prevented medin-induced EC death likely through Nrf2-dependent upregulation of antioxidant stress response (Karamanova N, et al. J Am Heart Assoc 2020; 9:e014810). Thus, it was tested whether medin aggravates EC hypoxic injury, and whether GM1L would prevent medin and/or hypoxic injury to ECs.

Methods. Recombinant medin was expressed in Lemo 21 (DE3) cells using pOPINS-medin (Karamanova N, et al. J Am Heart Assoc 2020; 9:e014810) and confirmed to have >95% purity by SDS-PAGE. GM1L was prepared from phosphatidylcholine, cholesterol and monosialoganglioside (molar ratios 70:25:5) using lipid hydration method (Karamanova N, et al. J Am Heart Assoc 2020; 9:e014810). Primary human brain microvascular ECs (Lonza, passages 4-8) were exposed for 20 hours to 4 different treatments (vehicle control, medin 5 μM, medin 5 μM+GM1L 300 μg/ml or GM1L 300 μg/ml) under two aerobic conditions: physoxia (5% oxygen) or hypoxia (1% oxygen). Cell viability was assessed using calcein-AM fluorescence (based on principle of intact esterases in viable cells that releases fluorescent calcein) using flow cytometry (measurements expressed relative to control ECs exposed to room air). Gene expression of antioxidant enzymes heme-oxygenase 1 (H01), NADPH quinone dehydrogenase (NQO1) and superoxide dismutase 1 (SOD1) were measured separately by qPCR on ECs.

Results. ECs treated with medin showed reduced viability under physoxic and more so under hypoxic condition; and treatment with GM1L prevented injury induced by hypoxic condition or medin treatment. ECs treated with GM1L showed upregulation of antioxidant stress enzymes HO-1, NQO1 and SOD1, while medin treatment did not elicit a change in antioxidant enzyme gene expression versus control.

Conclusions. Medin exposure showed significant additive adverse effect on viability of human brain microvascular endothelial cells exposed to hypoxic insult compared to medin alone. Because medin is associated with aging vasculature, and aging is associated with ischemic injury from atherosclerotic cerebrovascular disease, medin may be an important modulator of injury in advanced cerebrovascular disease and a treatment target. GM1-containing nanoliposomes prevented endothelial cell hypoxic injury as well as medin-mediated cell injury, likely through effects on upregulation of antioxidant defense mechanisms.

### Example 2: NLGM1 Prevents Hypoxic/Ischemic Injury to Vascular Cells

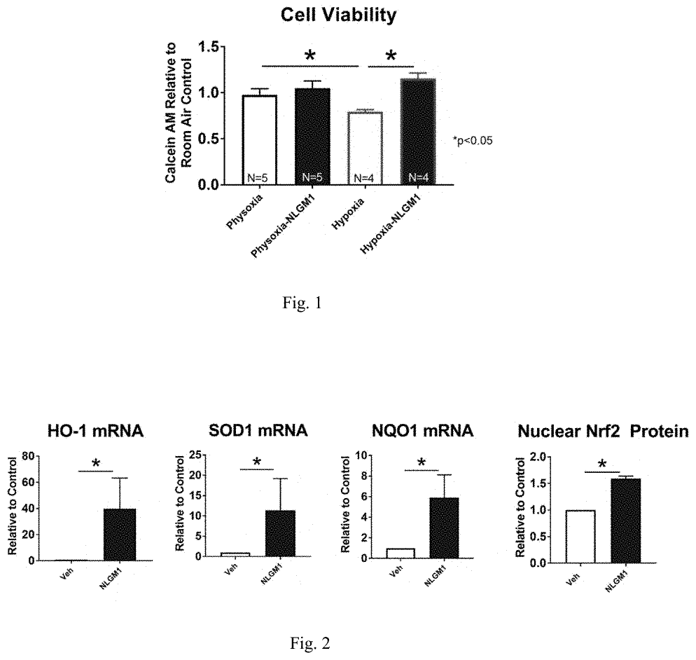

Human brain microvascular endothelial cells (HBMVECs) were exposed to physoxia (normal tissue oxygen level, 5%), and hypoxia (1% oxygen) for 20 hours without and with the nanoliposome, NLGM1, composed of phosphatidylcholine, cholesterol and monosialoganglioside at molar ratios 70:25:5, respectively, 300 μg/mL) (FIG. 1). Cell viability was assessed using calcein-AM fluorescence using flow cytometry. Measurements are expressed relative to control HBMVECs exposed to room air. Hypoxia caused significant reduction in cell viability. Treatment with NLGM1 protected against hypoxia/ischemia mediated endothelial cell/vascular cell death.

### Example 3: NLGM1 Increased Gene Expression of Antioxidant Stress Enzymes in Endothelial Cells, Mediated Through Nrf2 Activation

Human endothelial cells exposed for 20 hours to NLGM1 (300 μg/mL) showed increased gene (FIG. 2) and protein expression of the antioxidant enzymes heme oxygenase-1 (HO-1), superoxide dismutase-1 (SOD-1) and NADPH quinone dehydrogenase 1 (NQO1). Quantification of nuclear factor erythroid 2-related factor 2 (Nrf2) in nuclear components of the endothelial cells show increased Nrf2 in NLGM1 treated cells versus controls. Nrf2 is a transcription factor that is a master regulator of production of antioxidant enzymes and protective mechanisms against hypoxic/ischemic stress and inflammatory stress.

### Example 4: NLGM1 Increased Endothelial Cell Production of Sequestasome (p62) and LC3 II

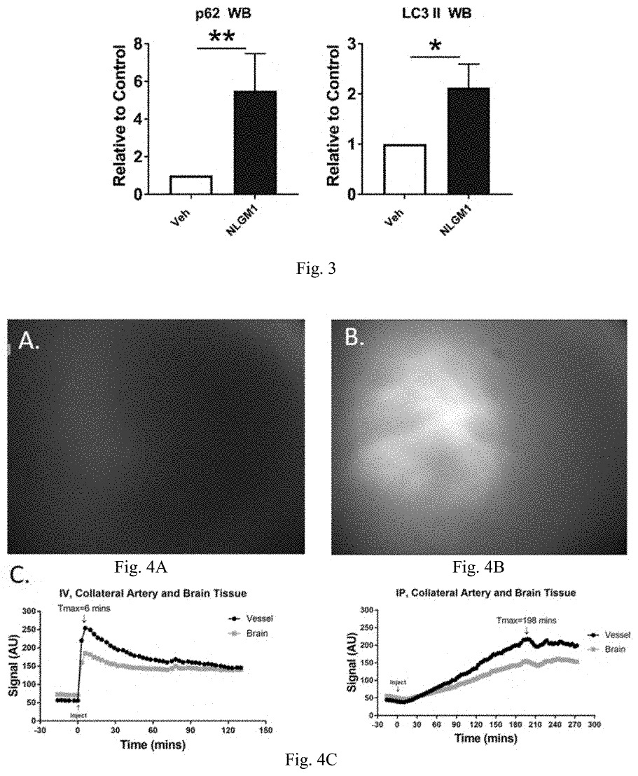

Treatment of human endothelial cells with NLGM1 (300 μg/mL, 20 hours) increased the protein expression of p62/sequestasome, a main regulator of the autophagic pathway that directs ubiquinated cargoes to autophagosomes for degradation (FIG. 3). NLGM1 treatment of endothelial cells also increased the protein expression of LC3-II, a standard marker for autophagosomes, and is specifically associated with autophagosomes and autolysosomes. These results suggest that another protective mechanism derived from NLGM1 treatment is enhancement of autophagy process.

### Example 5: NLGM1 Given to Mice Intravenously and Intraperitoneally Show Good Intravascular Bioavailability and Reaches and Persists in Cerebral Circulation

The pharmacokinetic properties of NLGM1 was tested in vivo in C56BL/6 mice by conjugating NLGM1 with a fluorescent marker. The mouse was imaged in vivo using a Miniscope imaging system that allowed fluorescent imaging of blood flow in cerebral vessels after Minsicope imaging system was incorporated through a cranial window. A single intravenous (IV) injection via tail vein was performed and imaging was done serially to measure fluorescent signal in cerebral vessel lumen and brain parenchymal tissue. Separate intraperitoneal (IP) injection was also done. FIGS. 4A-B shows brain fluorescent signal just before IV injection of NLGM1 (A) and at peak signal (B) showing delivery of NLGM1 to cerebral vessels and presence of signal in brain parenchyma. FIG. 4C shows the timeplot of fluorescent signal for IV and IP injections showing peak signal at 6 mins (IV) and 198 mins (IP) and persistence of signal (NLGM1 circulation) for >2 hours for single IV injection and >4 hours for single IP injection. These data show the persistence of NLGM1 in cerebral circulation with a single injection.

### Example 6: NLGM1 Reduced Neurologic Damage and Infarct Size in Mice Exposed to Stroke Via Middle Cerebral Artery Occlusion

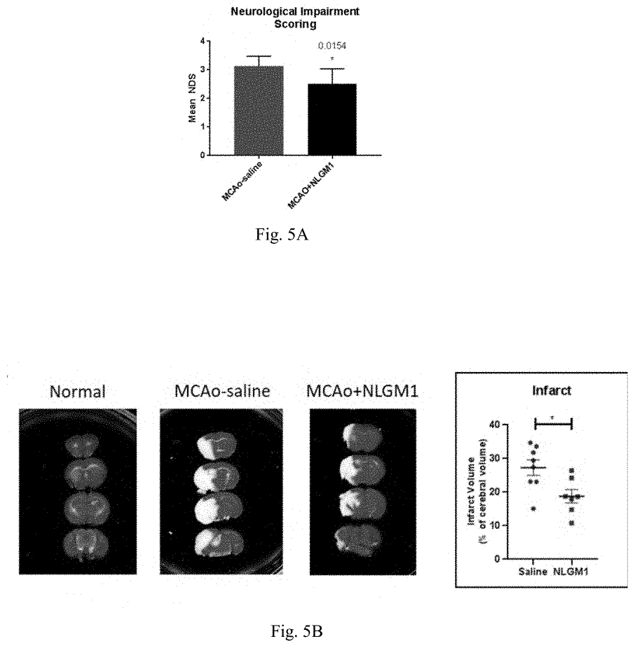

Wild type C57BL/6 mice and induced stroke were tested using middle cerebral artery occlusion for 45 minutes followed by release of occlusion (ischemia-reperfusion injury). One set of mice (control group) received saline and another set of mice received NLGM1 (PC:chol:GM1 at 70:25:5 molar ratios, 10,000 μg/mL given prior to arterial occlusion, prior to reperfusion and after reperfusion, IV and IP combination, total of 4 doses). The animals underwent neurologic impairment scoring after 24 hours followed by sacrifice to measure brain infarct size using TTC staining. Results shown in FIG. 5 showed significant reduction in neurologic impairment scoring in NLGM1-treated mice undergoing MCAo occlusion versus saline-treated controls. Quantification of brain infarct size showed significant reduction (˜33% relative reduction, ˜10% absolute reduction) of infarct size in NLGM1-treated mice versus saline-treated controls.

### Example 7: Nanoliposomes Reduce Stroke Injury Following Middle Cerebral Artery Occlusion in Mice

Neuroprotective strategies for stroke remain inadequate. Nanoliposomes comprising phosphatidylcholine, cholesterol and monosialogangliosides (NL) induced an antioxidant protective response in endothelial cells exposed to amyloid insults. It was tested whether NL will preserve SH-SY5Y neuroblastoma cell viability following hypoxic injury and will reduce injury in mice following middle cerebral artery occlusion (MCAO).

Methods: Neuroblastoma were exposed to 20-hour physoxic (5% oxygen) or hypoxic (1% oxygen) condition without or with NL (100 or 300 μg/mL). Viability was measured using calcein-acetoxymethyl fluorescence and SH-SY5Y gene expression of antioxidant proteins heme oxygenase-1 (HO-1), NAD(P)H quinone dehydrogenase 1 (NQO1) and superoxide dismutase 1 (SOD1) were measured by quantitative polymerase chain reaction. C57BL/6J mice were treated with saline (N=8) or NL (20000 ug/mL, N=7) while undergoing 60-minute MCAO followed by reperfusion. Day 2 post-injury neurologic impairment score and infarction size were compared.

Results: Neuroblastoma showed reduced viability following hypoxia that was reversed by NL. NL increased gene expression of HO-1, NQO1 and SOD1 versus controls. NL-treated mice showed reduced neurologic impairment and brain infarct size (18.8±2% versus 27.3±2.3%, p=0.017) versus controls.

Conclusions: NL reduced stroke injury in mice subjected to MCAO likely through induction of an antioxidant stress response. NL can be used in the treatment of stroke.

Methods. Nanoliposomes. NL was prepared from phosphatidylcholine, cholesterol and monosialoganglioside (70:25:5% molar ratios, Avanti, Alabaster Ala.) using lipid film hydration4, 5. Hydrodynamic diameters were measured by dynamic light scattering and zeta potential by electrophoretic light scattering.

Hypoxia. SH-SY5Y human neuroblastoma cells (ECACC, Public Health England, passages 16-18) were exposed (20-hours) to one of the following conditions using BioTek-Cytation 5 (Fisher-Scientific, Waltham Mass.): physoxia (5/5/90% oxygen/carbon dioxide/nitrogen), hypoxia (1/5/94% oxygen/carbon dioxide/nitrogen), hypoxia treated with NL (100 μg/mL or 300 μg/mL, the latter dose previously reported to confer endothelial cell protection (Franco D A, et al. J Am Heart Assoc. 2016; 5; and Karamanova N, et al. J Am Heart Assoc. 2020; 9:e014810). Cells were given calcein-acetoxymethyl (10 nmol/L, Life Technologies, measure of cell viability through detection of active/intact esterases (Karamanova N, et al. J Am Heart Assoc. 2020; 9:e014810) and viability was measured using flow cytometer (Beckman-Coulter FC500, Indianapolis Ind., 494/517 nm excitation/emission) (Karamanova N, et al. J Am Heart Assoc. 2020; 9:e014810). Separate cells were exposed to same treatment conditions and HO-1, SOD1 and NQO1 gene expressions measured (Karamanova N, et al. J Am Heart Assoc. 2020; 9:e014810). Human brain microvascular endothelial cells (CellBiologics, Chicago Ill., passages 5-8) were exposed for 20-hours to hypoxia (1% oxygen) without or with NL (100 or 300 μg/ml) and viability was compared with control cells in incubator (room air) using calcein-acetoxymethyl fluorescence.

NL Injection. To test delivery/persistence of NL, a fluorophore-containing NL modification (phosphatidylcholine, monosialoganglioside, 1,2-dioleoyl-sn-glycero-3-phosphoethanolamine-N-carboxyfluorescein in 85:5:10% molar ratios) was produced. Three C57BL/6J male mice (8-weeks old, Jackson Laboratory, Bar Harbor Me.) underwent surgery to secure a V3 Miniscope to the skull (Ghosh K K, et al. Nat Methods. 2011; 8:871-8). Baseline images were acquired and then 0.1 ml of NL (10000 μg/ml) intravenous (IV) injection was given through tail vein. After 1-day recovery, the process was repeated post-injection of 0.1 ml of NL via intraperitoneal (IP) injection. Fluorescent signals from 2 regions of interest (cerebral artery and brain parenchyma) were measured using ImageJ (National Institutes of Health, Bethesda Md.).

Nanoliposome production. NL was prepared from phosphatidylcholine, cholesterol and monosialoganglioside (70:25:5% molar ratios) using lipid film hydration method. The hydrodynamic diameters of the liposomal nanoparticles were measured by dynamic light scattering in triplicates using a Nano ZS Zetasizer (Malvern Panalytical, Westborough Mass.) at 25° C. In this study, the size reported is based on average particle diameter by volume. The zeta potential values of the NLGM1 suspensions were measured in disposable cuvettes equipped with gold electrodes. Zeta potential was determined using electrophoretic light scattering and is reported as the Z-average. Each measurement was performed on freshly prepared samples without dilutions.

Gene Expression Measurements. Gene expression was measured following lysis, ribonucleic acid extraction and conversion to complementary deoxyribonucleic acid using Aurum Total RNA Mini Kit and iScript cDNA synthesis kit (Bio-Rad Laboratories, Hercules Calif.). Primers for HO-1 (SEQ ID NO: 1; F-5′-GAAGACACCCUAAUGUGGCAGCTG-3′; SEQ ID NO: 2; R-5′-CAGCUGCCACAUUAGGGUGUCUUCCAG-3′; SEQ ID NO: 3; F-5′-ACAACAUUGUCUGAUAGUAGCUUGA-3′; SEQ ID NO: 4 R-5′-UCAAGCUACUAUCAGACAAUGUUGUUU-3′) and SOD1 (SEQ ID NO: 5 F-5′-CCTCGGAACCAGGACCT-3′; SEQ ID NO: 6 F-5′-TTAATGCTTCCCCACACCTT-3′) were obtained from IDT DNA Technologies (Coralville Iowa) and NQO1 (SEQ ID NO: 7; F-5′-ATGTATGACAAAGGACCCTTCC-3′; and SEQ ID NO: 8 R-5′-TCCCTTGCAGAGAGTACATGG-3′) was obtained from Sigma-Aldrich. β-actin served as reference normalization gene.

Miniscope animal imaging. Three C57BL/6J male mice (8-weeks old, (Jackson Laboratory, Bar Harbor Me.) underwent surgery to secure a V3 Miniscope to the skull. Rats were anesthetized with isoflurane (induction 5% at 1 L/min for 5 minutes and maintained at 2% at 1 L/min for duration of surgery). A single GRIN lens (Edmund Optics, #64-538) was implanted perpendicular to the parieto-temporal cortex. Seven days post-surgery, the mouse was anesthetized and secured in a stereotaxic frame for imaging. Imaging with modified Miniscope software was done with identical capture settings (Achromatic lens: 7.5 mm, FPS: 30, Exposure: 100%, Gain: 0, LED: 100%) capturing continuous video (30 Hz) modified to capture time lapse video (1 image per ˜1-5 minutes) turning the LED to 0% between image acquisitions. After surgery, topical lidocaine and bacitracin was applied around the implanted dental acrylic cap.

Middle cerebral artery occlusion surgery. Rats were anesthetized with isoflurane (3% for induction and 1.5% during the rest of the surgical procedure mixed with 30% oxygen and 70% nitrogen). A silicon-coated 6-0 nylon suture was introduced into the external carotid artery and advanced up to the internal carotid artery to occlude the middle cerebral artery for 60 minutes followed by removal of the filament to restore perfusion. Post-operative care included administration of antibiotics (Covenia 8 mg/kg, subcutaneous) and analgesic (buprenorphine sustained release 0.05 mg/kg, subcutaneous). The animals were recovered in pre-warmed cage with access to food and water and monitored until they were conscious and ambulatory whereupon they were returned to their home cage. They were assessed daily for pain and discomfort until the experimental endpoints.

Neurologic scoring and infarction volume. Neurological score was measured using a 5-point scale (0—No neurologic deficit; 1—Failure to extend left forepaw fully; 2—Circling to the left indicates a moderate focal neurologic deficit; 3—Falling to the left shows severe focal deficit; 4—Inability to walk spontaneously and diminished level of consciousness). Mice underwent deep anesthetization with isoflurane (2-3%) and killed by cervical dislocation. Brain coronal sectioning (2 mm thickness) was done using a mouse brain matrix (Roboz Surgical Instrument, Gaithersburg Md.). Brain coronal slices (2 mm) were stained with 1% 2,3,4-triphenyl tetrazolium chloride (Sigma-Aldrich, 37±0.5° C., 15-20 minutes).

Sample size calculations. Animal sample size consideration for stroke outcome was based on comparison of two means, with assumed infarct size of 30% (standard deviation of 4.8%) in control mice (Zhao X J, et al. PLoS One. 2017; 12:e0180822) and assumed 28% reduction with NL, requiring N=7 per group (α=0.05, power=0.8).

Middle cerebral artery occlusion injury. Twenty-week old male C57BL/6J mice underwent transient MCAO (Zhao X J, et al. PLoS One. 2017; 12: e0180822). A silicon-coated 6-0 nylon suture was introduced to occlude the middle cerebral artery for 60-minutes followed by filament removal to restore perfusion. Mice were randomly assigned to either saline (N=8) or NL (N=7, 10000 μg/ml) intraperitoneally (IP) 1-hour prior to occlusion and intravenously (IV) just before reperfusion.

Neurological deficit scoring (NDS) used modified Bederson scale 48-hours post-stroke (Zhao X J, et al. PLoS One. 2017; 12:e0180822) followed by sacrifice. Brain coronal slices (2 mm) were stained with 1% 2,3,4-triphenyl tetrazolium chloride (Sigma-Aldrich). Using ImageJ, corrected infarct volume was calculated (Zhao X J, et al. PLoS One. 2017; 12:e0180822). An algorithm (McBride D W, et al. Transl Stroke Res. 2015; 6:323-38) was applied to indirectly calculate infarct volumes, corrected for edema, and brain edema. Measurements were performed by an investigator blinded to treatment allocation.

Data Analyses. Cell data were compared using one-way repeated measures analysis of variance (RM-ANOVA) with Holm-Sidak pairwise comparison (normally distributed) or RM-ANOVA on ranks with Tukey pairwise comparison (non-normal distribution/unequal variance) (Sigma Stat 3.5, Systat, San Jose Calif.). Animal outcomes were compared using unpaired Student's t-test. Significant p-value was set at 0.05 (two-sided). Data are presented as mean±standard error of means.

Results. Nanoliposomes were 38.83±1.23 nm in size with polydispersity index of 0.32 and Z-potential of −8.76 mV.

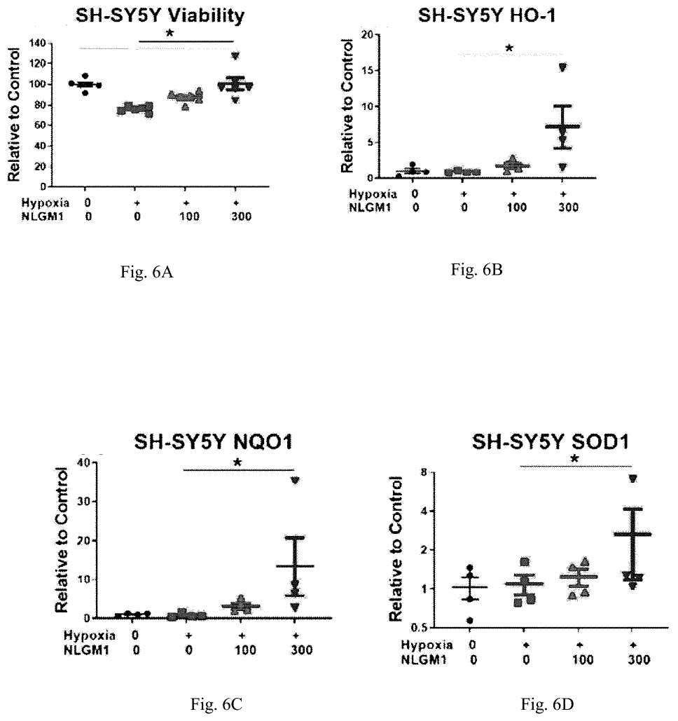

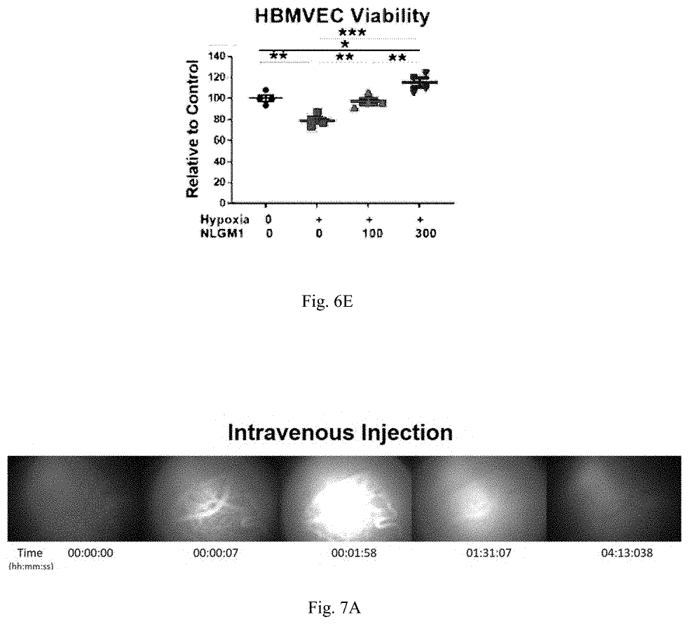

Neuroblastoma exposed to hypoxia had reduced viability versus control, while treatment with 100 or 300 μg/ml NL restored cell viability (FIG. 6). There was no difference in HO-1, NQO1 or SOD1 gene expression between neuroblastoma exposed to hypoxia versus physoxia, but treatment with NL (300 μg/ml) increased HO-1, NQO1 and SOD1 gene expressions in hypoxic condition. Endothelial cells had reduced viability following hypoxia, with viability restored by NL treatment.

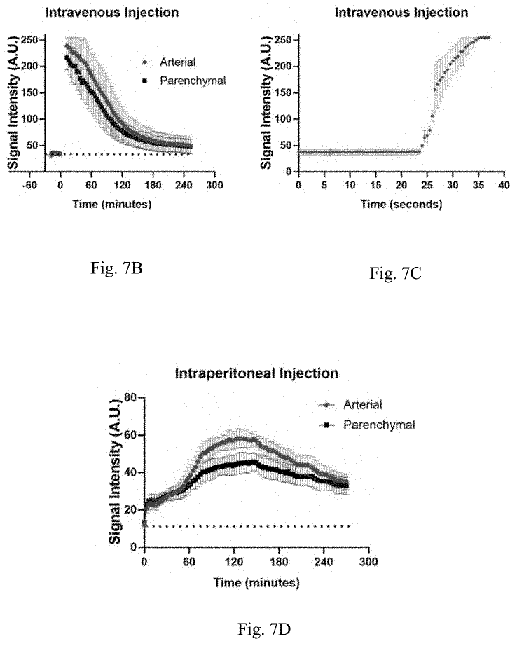

There was persistent brain fluorescent NL signal up to 2 hours post-IV and more than 4 hours post-IP NL injections (FIG. 7).

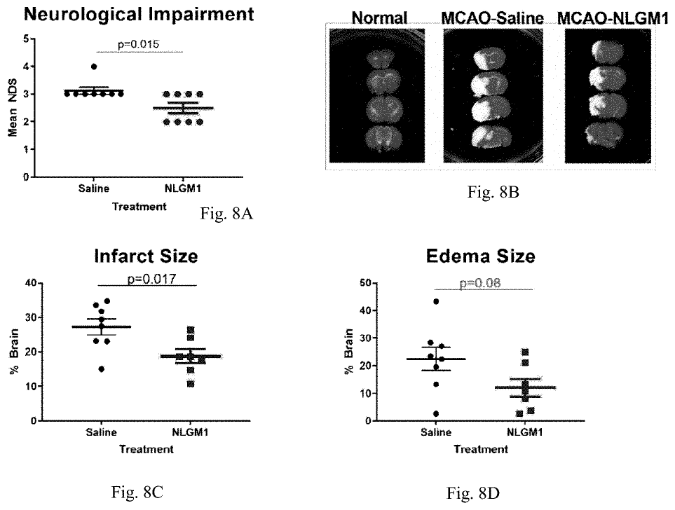

Mice given NL showed improved neurologic impairment score versus saline control (FIG. 8). They also had significantly smaller infarcts and a trend towards decreased brain edema.

Discussion One person dies from stroke every 4 minutes in the U.S. (Nour M, et al. Interv Neurol. 2013; 1:185-99). Neutralizing oxidative stress is a adjuvant strategy to reperfusion as the ischemic brain is susceptible to oxidative damage due to high oxygen consumption and low antioxidant capacity (Chamorro A, et al. Lancet Neurol. 2016; 15:869-881). NL prevented endothelial dysfunction and cell death induced by amyloidogenic light chain and aging-associated medin proteins (Franco D A, et al. J Am Heart Assoc. 2016; 5; and Karamanova N, et al. J Am Heart Assoc. 2020; 9:e014810). The results show that NL also preserved viability of cells exposed to hypoxia. NL's protective effect was attributed to increased gene/protein expression of endogenous antioxidants HO-1, NQO1 and SOD1 through Nrf2-mediated signaling (Franco D A, et al. J Am Heart Assoc. 2016; 5; and Karamanova N, et al. J Am Heart Assoc. 2020; 9:e014810). As described herein, NL-treated neuroblastoma exposed to hypoxia increased these antioxidant enzymes and had preserved cell viability. In vivo, NL treatment reduced functional impairment and infarct size post-MCAO/reperfusion. The protective effect of NL following stroke is likely due to its ability to protect neuronal and endothelial cells against hypoxic insult. Miniscope imaging showed that NL persists in cerebrovascular circulation 2 hours or longer post-IV or post-IP administration, demonstrating sufficient exposure time within the therapeutic window for stroke.

The data are consistent with prior reports of mitigation of stroke injury by monosialoganglioside in preclinical models (Li L, et al. PLoS One. 2016; 11:e0144219), although monosialoganglioside had equivocal results in human clinical trials (Candelise L and Ciccone A. Cochrane Database Syst Rev. 2001:CD000094; and Zhang W, et al. Cell Transplant). The study described herein differed from the previous reports by injecting phospholipids in nanoliposomal structures, and NL was composed of 3 phospholipids, with monosialoganglioside comprising just 5% molar weight. The prior study (Li L, et al. PLoS One. 2016; 11:e0144219) used 150 mg/kg of monosialoganglioside resulting in 6.8% reduction whereas the NL used herein comprised ˜8.4 mg/kg monosialoganglioside resulting in 8.5% reduction in infarct volume. The prior study injected free monosialoganglioside in saline; due to its low solubility in aqueous medium this glycolipid forms micellar aggregates the sizes of which were not reported.

The NL timing employed here can be translatable to strokes with prodromal transient ischemic attacks wherein NL can be given before complete occlusion onset.

Phosphatidylcholine, cholesterol and monosialoganglioside NL prevented hypoxia-induced injury in neuroblastoma and endothelial cells and reduced functional and structural brain damage in mice exposed to MCAO stroke.

1. A method of treating stroke, the method comprising administering to a subject a therapeutically effective amount of a composition comprising a nanoliposome, wherein the nanoliposome comprises a phospholipid, cholesterol, and a glycosphingolipid moiety.

2. A method of reducing or preventing endothelial cell or vascular hypoxic/ischemic injury in a subject, the method comprising administering to the subject a therapeutically effective amount of a composition comprising a nanoliposome, wherein the nanoliposome comprises a phospholipid, cholesterol, and a glycosphingolipid moiety.

3. A method of reducing or preventing medin-mediated injury in a subject, the method comprising administering to the subject a therapeutically effective amount of a composition comprising a nanoliposome, wherein the nanoliposome comprises a phospholipid, cholesterol, and a glycosphingolipid moiety.

4. (canceled)

5. The method of claim 1, wherein the subject has ischemic cerebrovascular disease.

6. The method of claim 1, wherein the subject has an ischemic injury caused by atherosclerotic cerebrovascular disease.

7. (canceled)

8. The method of claim 1, wherein the subject does not have increased or elevated levels of medin.

9. The method of claim 1, wherein the phospholipid is phosphatidylcholine or phosphatidic acid.

10. The method of claim 1 The method of any of the preceding claims, wherein the glycosphingolipid moiety is a cerebroside, ganglioside or a globoside.

11. The method of claim 10, wherein the glycosphingolipid moiety is a ganglioside.

12. The method of claim 11, wherein the ganglioside is GM1, GM2, GM3, GD1a, GD1b, GD2, GD3, GT1b, GT3 or GQ1.

13. The method of claim 12, wherein the ganglioside is monosialoganglioside (GM1).

14. The method of claim 1, wherein the phospholipid, cholesterol, and glycosphingolipid moiety are present in a molar ratio of 70:25:5, respectively.

15. The method of claim 13, wherein the composition comprises phosphatidylcholine, cholesterol, and monosialoganglioside (GM1) in a molar ratio of 70:25:5, respectively.

16. The method of claim 1, wherein the administration of the composition increases one or more antioxidant enzymes.

17. The method of claim 1, wherein the administration of the composition increases one more or transcription factors.

18. The method of claim 1, wherein the administration of the composition increases nitric oxide bioavailability.

19. The method of claim 1, wherein the administration of the composition reduces one or more proinflammatory cytokines or one or more prothrombotic cytokines.

20. The method of claim 19, wherein the one or more proinflammatory cytokines are IL-1β, IL-8, TNF-α or IL-6.

21. The method of claim 19, wherein the one or more prothrombotic cytokines are ICAM-1, VCAM-1, MCP, caspase-1 or PAI-1.

22. The method of claim 16, wherein the one or more antioxidant enzymes are heme-oxygenase 1 (HO-1), NADPH quinone dehydrogenase (NQO1), superoxide dismutase 1 (SOD1), catalase, glutathione peroxidase, peroxiredoxin I and II, thioredoxin, myeloperoxidase, thioredoxin reductase, or a combination thereof.

23. The method of claim 17, wherein the one or more of the transcription factors is nuclear factor erythroid 2-related factor 2 (Nrf2).

24. (canceled)

25. The method of claim 1, wherein the composition further comprises a therapeutic cargo.

26. The method of claim 25, wherein the therapeutic cargo is clusterin, apolipoprotein J, apolipoprotein E, an antibody fragment, an aptamer, a small interfering RNA (siRNA), or a short hairpin RNA (shRNA).

27. The method of claim 26, wherein the clusterin is secretory clusterin (sCLU).

28. (canceled)

29. The method of claim 1, wherein the subject is identified in need of treatment before the administration step.

30. The method of claim 1, wherein the subject is identified as not having an increased level of medin protein compared to a control subject.

31. (canceled)
